(19) United States
(12) Patent Application Publication
(10) Pub. No.: US 2025/0082251 A1
(43) Pub. Date: Mar. 13, 2025

(54) METHOD AND SYSTEM FOR NOISE FILTERING FROM ECG SIGNALS

(71) Applicant: West Affum Holdings DAC, Dublin (IE)

(72) Inventor: Joseph L Sullivan, Kirkland, WA (US)

(73) Assignee: West Affum Holdings DAC, Dublin (IE)

(21) Appl. No.: 18/404,963

(22) Filed: Jan. 5, 2024

Related U.S. Application Data
(60) Provisional application No. 63/537,404, filed on Sep. 8, 2023.

(51) Int. Cl. A61B 5/366; A61B 5/00; A61B 5/256; A61B 5/282; A61B 5/352

(52) U.S. Cl. CPC A61B 5/366; A61B 5/256; A61B 5/282; A61B 5/352; A61B 5/7203; A61B 5/725; A61B 5/7264

(57) ABSTRACT

A wearable medical system (WMS) filters noise from one or more ECG signals. The WMS includes a plurality of ECG electrodes to sense the one or more ECG signals of a patient, and a processor in communication with the plurality of ECG electrodes. The processor is configured to receive the one or more ECG signals, sample the one or more ECG signals at a predefined frequency, determine if a measurement of samples exceeds a noise threshold, and provide a blanking period if the measurement of the samples exceeds the noise threshold. The blanking period replaces the samples in the blanking period with a not a number (NaN) value, thereby keeping the samples set in time.

### CROSS-REFERENCE TO RELATED APPLICATIONS

This application claims priority to and the benefit of the provisional patent application No. 63/537,404 titled “METHOD AND SYSTEM FOR NOISE FILTERING FROM ECG SIGNALS TO DETECT ATRIAL FIBRILLATION”, filed in the United States Patent and Trademark Office on Sep. 8, 2023. The specification of the above referenced patent application is incorporated herein by reference in its entirety.

### TECHNICAL FIELD

The present technology relates to a wearable medical system and more particularly, but not by way of limiting, the present technology relates to filtering noise present in the Electrocardiogram (ECG) signal for a medical monitoring device.

### BACKGROUND

Conventionally, medical monitoring devices such as ECG analysis devices are configured to perform rhythm analysis using an ECG signal of a patient. A result of the rhythm analysis may include detection of atrial arrhythmias such as atrial fibrillation (AF), ventricular arrhythmias such as ventricular fibrillation (VF) or ventricular tachycardia (VT), and the like. The ECG analysis devices analyze various features of the ECG signal like frequency, amplitude, slope or wave morphology, among others.

Filters included in the ECG analysis device may check for QRS-like signals, radio transmission, cycle interference, and electrode contact during the rhythm analysis. However, errors such as presence of noise can cause confusion for an ECG analysis algorithm and interfere with the correct detection of arrhythmias. The incorrect detection may further lead to wrong decisions, thereby impacting the accuracy of rhythm analysis. Due to the lack of accurate determination of the true arrhythmias, the patient may be exposed to life-threatening situations.

### SUMMARY

The present disclosure relates to a wearable medical system (WMS) for filtering noise from one or more ECG signals. The wearable medical system includes a plurality of ECG electrodes to sense the one or more ECG signals of a patient and a processor in communication with the plurality of ECG electrodes. The processor is configured to receive the one or more ECG signals and sample the one or more ECG signals at a predefined frequency, the predefined frequency may be about 500 Hz. The processor is further configured to determine if a measurement of samples exceeds a noise threshold. For example, the measurement of samples includes a first measurement and a second measurement. The first measurement being an absolute value of an amplitude difference between consecutive samples (i.e. the slope) that is determined to exceed a first threshold, and the second measurement being an absolute value of an amplitude difference between a first sample and a last sample in a series of samples that is determined to exceed a second threshold. As an example, the first threshold is about 2 mV/sample and the second threshold is about 5 mV.

The processor is further configured to provide a blanking period if the slope of the measurement of the samples exceeds the noise threshold. In some embodiments, the blanking period is implemented by a bi-directional filter configured to blank samples before and after the consecutive samples that exceed the first threshold or the series of samples that exceed the noise threshold. The blanking period replaces the samples in the blanking period with a not a number (NaN) value, thereby keeping the samples set in time.

The processor may be further configured to blank ECG signals having a duration of less than 30 seconds between two blanking periods. The WMS further includes a filter configured to filter the ECG signals and upon filtering, the processor is further configured to determine a heart rate based on an R-R interval of QRS complexes detected from the ECG signals.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates an example of a wearable medical system (WMS) worn by a patient, according to an embodiment of the present disclosure.

FIG. 2 illustrates a block diagram of a wearable monitoring device (WMD), according to an embodiment of the present disclosure.

FIG. 3 illustrates a block diagram that depicts communication between the WMD and a remote location, according to an embodiment of the present disclosure.

FIG. 4 illustrates a block diagram that depicts communication between the WMD and the remote location, according to another embodiment of the present disclosure.

FIGS. 5A and 5B illustrate graphs depicting the input to and output from a noise filter module of the WMD, according to an embodiment of the present disclosure.

FIG. 6 illustrates a graph that depicts identification of dominant and non-dominant beats and R-R interval between consecutive dominant beats on a single channel of a plurality of channels, according to an embodiment of the present disclosure.

FIG. 7 illustrates a graph that depicts identification of the dominant and the non-dominant beats in a noisy signal and R-R interval between the consecutive dominant beats, according to an embodiment of the present disclosure.

FIG. 8 illustrates an example method for filtering noise from one or more ECG signals using the WMD, according to an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures or methods associated with the wearable medical system have not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one aspect” or “an aspect” means that a particular feature, structure, or characteristic described in connection with an embodiment is included in at least one aspect. Thus, the appearances of the phrases “in one aspect” or “in an aspect” in various places throughout this specification are not necessarily all referring to the same embodiment. Furthermore, the particular features, structures, or characteristics may be combined in any suitable manner in one or more aspects.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

During the rhythm analysis performed by the ECG analysis devices, various features of the ECG signal are analyzed to detect abnormalities or corruption by noise. As a non-limiting example, based on analyzing the R-R intervals, R-R variability of the ECG signals may be determined which aids in the detection or determination of the arrhythmia such as AF. The determination of the R-R intervals may consider unwanted beats corresponding to QRS complexes detected in the ECG signal that may not help detect the AF accurately. The beats may include at least one of: a non-supraventricular beat such as a premature ventricular contraction, noise incorrectly detected as a beat, or a local peak in an ECG signal incorrectly detected as an R-wave such as but not limited to double counting. The usage of unwanted beats for the AF detection may degrade the performance of the ECG analysis device like an arrhythmia detector.

Further, the ECG analysis devices may be coupled to dry electrodes that may obtain signals that are more noise-prone than ECG monitors using adhesive electrodes. The noise can interfere with the detection of the AF causing R-R variability that may be confused with the actual AF.

As another non-limiting example, noise can also interfere with the detection of other arrhythmias such as VF. VF is a random signal and noise may be difficult to distinguish from a true cardiac VF. Also, periods of noise may interfere with the detection of the QRS complexes, which complicates the heart rate calculation and detection of many arrhythmias. If the noise is periodic, the noise may dominate the ECG signals and obscure the true QRS complexes. Thus, the presence of noise also may interfere with the assessment of the R-R interval, thereby reducing the accuracy of the arrhythmia detector. As yet another non-limiting example, noise can also interfere with the detection of VT. Usually, ECG analysis device will deliver a shock when VT is detected if the rate exceeds a preset value. Additionally, waveform and energy levels for terminating the detected arrhythmia need to be monitored for proper rhythm analysis.

Further, if the noise is not filtered out during the rhythm analysis, it may tend to erroneously increase the detected heart rate and the ECG analysis algorithm may mistake noise for QRS complexes that may result in anomalous QRS related measurements. Thus, it may be useful to detect and filter noise from ECG signals.

Generally, due to the presence of noise, the ECG analysis algorithm may not detect arrhythmias correctly and/or may inadvertently cause the respective ECG analysis devices with shock capability to deliver either inappropriate shocks or completely fail to deliver a shock to the patient(s). Moreover, failure to follow recommended operating procedures such as avoidance of patient movement or manufacturer's instructions for the respective ECG analysis device may result in inappropriate shocks or failure to shock.

The embodiments of the present disclosure disclose various modules that avoid potentially wrong decisions that the ECG analysis algorithm (or the ECG analysis device) may undertake due to the presence of noise samples during the rhythm analysis. The detected noise samples are replaced with placeholders that have no ECG value and the ECG analysis algorithm is designed to ignore the placeholder samples. As a result, the ECG analysis algorithm may still analyze portions of the ECG signal that contain legitimate ECG samples, thereby aiding in noise filtering from ECG signals.

Specifically, the present disclosure relates to wearable medical systems that detect cardiac rhythm disorders in a patient. Such systems include at least a wearable monitoring device (WMD) that receives one or more electrocardiogram (ECG) signals from multiple channels and filters noise present in the received one or more ECG signals. The WMD selects at least two channels of the multiple channels, after filtering the noise, from which the ECG signals may be utilized for further operations. The WMD also selects beats corresponding to the ECG signals that may be used for determining R-R interval and Heart Rate (HR) variability. Further, the WMD utilizes the R-R interval and the HR variability from the ECG signals corresponding to the selected channels for accurate determination of arrhythmias including at least atrial fibrillation (AF).

The embodiments disclosed herein facilitate application of noise detection and blanking to any WMD or ECG analysis device that performs rhythm analysis. Blanking periods of noise avoids incorrect rhythm analysis results and caters to the need of performing analysis on the clean ECG signals as set forth herein.

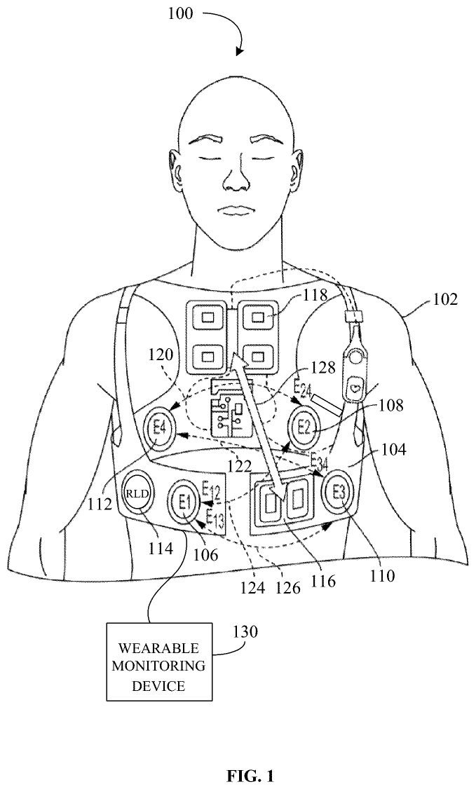

FIG. 1 illustrates an example of a wearable medical system (WMS) 100 worn by a patient 102, according to an embodiment of the present disclosure. The WMS 100 at least monitors electrocardiogram (ECG) signals of the patient 102, also referred to as a user 102. The patient 102 may be at least one of ambulatory patient or essentially bed-ridden patient. In an example, the user 102 may be a clinician including, but not limited to, a doctor, nurse, emergency medical technician (EMT), or other similarly tasked individuals or groups of individuals. In another example, the user 102 may be a bystander who might offer assistance or a trained person. In yet another example, the user 102 may be a remotely located and trained caregiver in communication with the WMS 100. Therefore, the terms “user” and “patient” may be used interchangeably throughout the disclosure and the particular context of these and other related terms within this description should be interpreted accordingly.

The WMS 100 includes a support structure 104 or a garment configured to be worn by the user 102 for one or more hours, days, or months. It will be understood that the support structure 104 is shown generically in FIG. 1 and FIG. 1 is provided to illustrate concepts about the support structure 104. However, it should be noted that the details disclosed in FIG. 1 are not to be construed as limiting with respect to how the support structure 104 is implemented, or how it is worn.

The support structure 104 may be implemented in a single component or a combination of multiple components. The support structure 104, in some embodiments, may include a vest, a half-vest, a garment, and the like, that may be worn similarly to analogous articles of clothing. In some embodiments, the support structure 104 may include a harness, one or more belts or straps, and the like, that allows the support structure 104 to be worn by the patient 102 around the torso, hips, over the shoulder, and the like. The one or more belts and straps are adjustable based on the physique of the patient 102. In some embodiments, the support structure 104 is waterproof. In some embodiments, the support structure 104 includes a container or housing, allowing the support structure 104 to be worn by the patient 102 using an adhesive material, for example as shown and described in U.S. Pat. No. 8,024,037 which is incorporated herein by reference in its entirety. The support structure 104 may be implemented as the support structure described in the U.S. Pat. Publication No. 2017/0056682 A1, which is incorporated herein by reference in its entirety. The person skilled in the art will recognize that additional components of the WMS 100 may be present in the container of the support structure 104 instead of being attached externally to the support structure 104, for example as described in the aforementioned '682 patent publication.

The support structure 104 comprises a plurality of ECG electrodes positioned on a front portion such as electrodes (E1) 106 and (E2) 108 and on a back portion such as an electrode (E3) 110 to sense one or more ECG signals and an electrode (E4) 112. Additionally, the support structure 104 includes a right-leg drive (RLD) electrode 114, also referred to as a common mode electrode, to manage common mode noise. The front portion of the support structure 104 further includes an anterior defibrillation electrode 116 and the back portion of the support structure 104 includes a posterior defibrillation electrode 118. The anterior defibrillation electrode 116 and the posterior defibrillation electrode 118 may be referred to as an anterior defibrillation pad 116 and a posterior defibrillation pad 118, respectively. The anterior defibrillation electrode 116 and the posterior defibrillation electrode 118 are collectively referred to as the defibrillation electrodes 116, 118.

The ECG electrodes (E1-E4), collectively referred to as ECG electrodes 106-112, may be placed circumferentially around the torso of the patient 102 so that the support structure 104 may be used to ensure adequate electrode-skin contact with the skin of the patient 102. It should be noted that alternative placement of the ECG electrodes 106-112 may be used, and the scope of the disclosed subject matter is not limited in this respect. For example, adhesive electrode embodiments may provide flexibility in electrode placement in selected locations of the body of the patient 102 and may achieve better signal pickup at the selected locations. For example, locations of the ECG electrodes 106-112 may be selected during a patient-fitting process in which various locations can be changed, and locations with better or best of the plurality of the ECG signals can be selected, although the scope of the disclosed subject matter is not limited in this respect.

In an example, four differential vectors may be formed by subtracting two digitized ECG signals. An ECG rhythm analysis may then be performed on the four differential vectors. The differential vectors include, for example, a vector (E24) 120, a vector (E34) 122, a vector (E12) 124, and a vector (E13) 126 that are derived from single-ended vectors. The WMS 100 may generate a defibrillator shock vector 128 between the anterior defibrillation electrode 116 and the posterior defibrillation electrode 118.

In some embodiments, the ECG signals from the four ECG electrodes 106-112 may be combined to form six different vectors. In some embodiments, the WMS 100 that monitors the ECG signals, also referred to as an ECG monitoring device 100, may use the four vectors i.e., E24 120, E34 122, E12 124, and E13 126, for QRS complex analysis or heart rate analysis to determine if a shock should be applied. Thus, the WMS 100 is capable of performing synchronous cardioversion as a therapy based on the heart rate analysis. In some embodiments, the WMS 100 is capable of performing the heart rate analysis and shock application determination if one or more of the above-mentioned vectors E24 120, E34 122, E12 124, and E13 126 are noisy or one or more of the ECG electrodes 106-112 or ECG leads are in a lead-off condition. The lead-off condition is a resultant of the ECG lead or at least one of the ECG electrodes 106-112 or the defibrillation electrodes 116, 118 not contacting the skin of the patient 102 or not sufficiently contacting the skin of the patient 102.

In some embodiments, at least three ECG electrodes of the ECG electrodes 106-112 may be used and corresponding three ECG vectors of the ECG vectors E24 120, E34 122, E12 124, and E13 126 may be analyzed. In some embodiments, five or six ECG vectors may be analyzed using all the ECG electrodes 106-112. In some embodiments, a single vector may be used and analyzed. It should be noted that, in general, the WMS 100 or the ECG monitoring device 100 may use and analyze fewer than four vectors or greater than four vectors. The number of vectors may be increased beyond six vectors by using additional ECG electrodes, and the scope of the disclosed subject matter is not limited in this respect.

In some embodiments, the WMS 100 may use four channels out of six possible differential channels formed from four independent electrodes placed around the chest of the patient 102. In some embodiments, the WMS 100 may use a different number of channels, including only one channel. In some embodiments, the WMS 100 may use a QRS detector of a single channel or multiple channels filtered ECG signal to detect a possible arrhythmia for full rhythm analysis. In some embodiments, where the ECG monitoring device comprises the WMS 100, a different number of ECG electrodes may be used, often a reduced number of electrodes or a different garment system may be used other than the support structure 104. In some embodiments, the ECG electrodes 106-112 may provide multiple vectors of the ECG signal and the QRS detector may operate on each of the multiple vectors.

The WMS 100, for example, is capable of excluding one or more of the vectors E24 120, E34 122, E12 124, and E13 126 that have noise or when a lead-off condition is detected. Monitoring four vectors rather than monitoring two vectors is believed to contribute to enhanced ECG signal analysis and processing of a shock application algorithm to reduce the number of false shock events.

In some embodiments, the WMS 100 is referred to as a cardiac monitoring device that uses the ECG electrodes 106-112 which may be configured to detect QRS complexes similar to QRS complexes that are normally conducted through an atrioventricular (AV) node. The QRS complexes conducted through the AV node can be referred to herein as “normally conducted QRS complexes.” The one or more ECG signals from the ECG electrodes 106-112 can be a mixture of multiple different QRS morphologies. Normally conducted QRS complex morphology identification may be used as described herein to determine the rhythms more accurately. The determination of rhythms include, but not limited to, determination of atrial fibrillation (AF) and the associated heart rate (HR) and HR variability, according to one or more embodiments. In some embodiments, the WMS 100 may solely detect the ECG signals from the ECG electrodes 106-112 and determine the presence of cardiac arrhythmia but may not provide any therapy, such as the application of shock or providing pacing pulses using the defibrillation electrodes 116, 118, also referred to as therapy electrodes 116, 118.

The ECG electrodes 106-112 attached to the support structure 104 may couple with a wearable monitoring device (WMD) 130 or a wearable cardiac monitor (WCM) 130 that is also attached to the support structure 104. The WMD 130 is configured to continuously monitor the ECG signals of the patient 102 and generate a rhythm discrimination. In some embodiments, the WMD 130 is configured to obtain the ECG signals of the patient 102. The ECG signals may be digitized by the WMD 130 for digital processing. In some embodiments, the WMD 130 is embedded in the WMS 100.

Within the context of the disclosed embodiments, it shall be noted that the ECG analysis device or WMD 130 may correspond to a hospital ECG monitor with rhythm alarms, a wearable monitor with arrhythmia detection, an automated external defibrillator (AED), a wearable cardioverter defibrillator (WCD), a Holter monitor, or other devices.

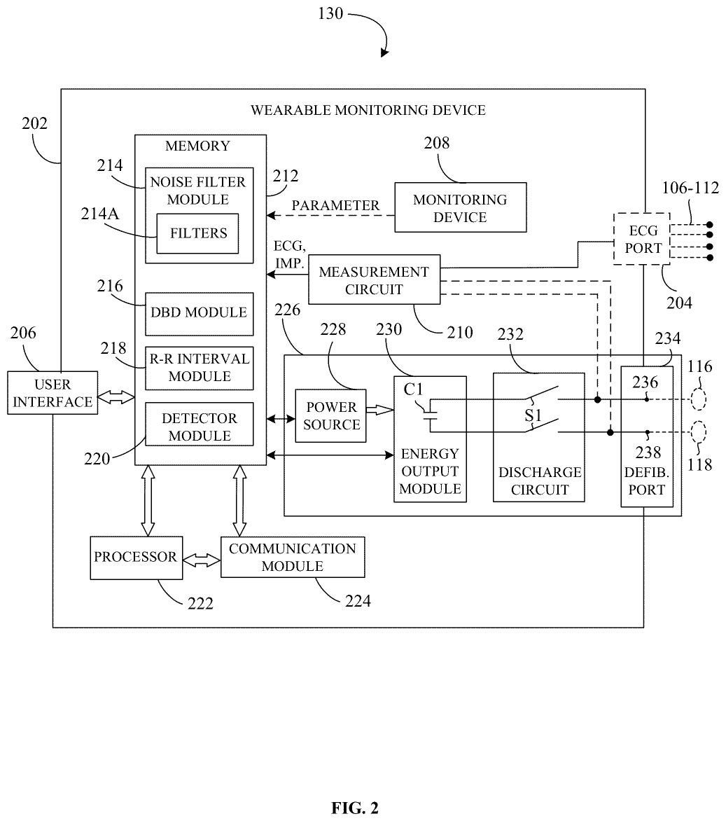

FIG. 2 illustrates the WMD 130 coupled to the WMS 100, as described in FIG. 1, according to an embodiment of the present disclosure. The WMD 130 detects and determines cardiac rhythm disorders and provides therapy based on the determination of the cardiac rhythm disorders. One or more components of the WMD 130 are provided in a housing 202, which is also referred to as a casing 202. The housing 202, in some embodiments, is the container described in FIG. 1. The components of the WMD 130, in some embodiments, include an ECG port 204, a user interface 206, a monitoring device 208, a measurement circuit 210, a memory 212, a processor 222, a communication module 224, and a therapy unit 226 coupled to the defibrillation electrodes 116, 118.

The user interface 206 may include one or more output devices, which may be visual, audible, or tactile, for communicating with the user 102, such as the bystander or physician, or providing human-perceptible indications (HPIs) by outputting images, sounds, vibrations, and the like. One of the one or more output devices further includes, for example, a light to indicate or a screen to display sensed, detected, and/or measured information by the WMD 130 and/or the ECG electrodes 106-112 and provide visual feedback to a rescuer for resuscitation attempts of the patient 102, as disclosed in FIG. 1. One of the one or more output devices, for an example, may be a speaker, which could be configured to issue voice prompts, beeps, loud alarm sounds, and/or words to warn bystanders, and the like.

The user interface 206 may further include one or more input devices that receive inputs from the patient 102 or the user 102. The one or more input devices may include various controls, such as push buttons, keyboards, touchscreens, one or more microphones, and the like. One of the one or more input devices may be a cancel switch, which is sometimes called an “I am alive” switch or “live man” switch as actuating the cancel switch by the patient 102 can prevent impending delivery of a shock to the patient 102.

The ECG port 204, also referred to as a sensor port 204, is coupled to or adapted for plugging in the ECG electrodes 106-112, also known as sensing electrodes 106-112 or the ECG leads. The ECG electrodes 106-112, for example, may be connected continuously to the ECG port 204. The WMD 130 may receive inputs from the ECG electrodes 106-112 through the ECG port 204. The ECG electrodes 106-112 are types of transducers that can sense an ECG signal, for example a 12-lead signal. In some embodiments, the ECG electrodes 106-112 can sense a signal from a different number of leads, especially if the ECG electrodes 106-112 make good electrical contact with the body of the patient 102 and particularly with the skin of the patient 102. The ECG electrodes 106-112 can be attached to the inside of the support structure 104, as disclosed in FIG. 1, for making good electrical contact with the patient 102.

The WMD 130 also includes a defibrillation port 234, in some embodiments, that may be a socket in the housing 202. The defibrillation port 234 may include electrical nodes 236, 238. Leads of the defibrillation electrodes 116, 118 may be plugged into the defibrillation port 234, to make electrical contact with electrical nodes 236, 238, respectively. In some embodiments, the defibrillation electrodes 116, 118 are connected continuously to the defibrillation port 234. The defibrillation port 234 may be used for guiding, via the defibrillation electrodes 116, 118, to the patient 102 at least some of electrical charge stored in the energy output module 230. The electric charge applied to the patient 102 is the shock for defibrillation, pacing, and the like. The defibrillation electrodes 116, 118 may be attached to the inside of the support structure 104 for making good electrical contact with the patient 102.

The WMD 130, according to some embodiments, also includes a fluid that can be deployed automatically between the ECG electrodes 106-112 and the skin of the patient 102. The fluid may be conductive, such as by including an electrolyte, for establishing better electrical contact between the ECG electrodes 106-112 and the skin of the patient 102. When the fluid is deployed, the electrical impedance between the ECG electrodes 106-112 and the skin is reduced. The fluid may be in the form of a low-viscosity gel that does not flow away from the ECG electrodes 106-112 after the fluid has been deployed. The fluid can be used for both the defibrillation electrodes 116, 118, and the ECG electrodes 106-112.

The fluid may be initially stored in a fluid reservoir (not shown in FIG. 2), coupled to the support structure 104 as disclosed in FIG. 1. Additionally, the WMD 130, for example, further includes a fluid deploying mechanism (not shown). The fluid deploying mechanism may be configured to cause at least some of the fluid to be released from the fluid reservoir and be deployed near one or more locations to which the ECG electrodes 106-112 are configured to be attached to the patient 102. In some embodiments, the fluid deploying mechanism is activated prior to the application of the shock, responsive to receiving an activation signal from the processor 222.

The monitoring device 208 is also referred to as an internal monitoring device 208 since the monitoring device 208 is incorporated within the housing 202. The monitoring device 208 may sense or monitor patient parameters such as physiological parameters of the patient 102, state parameters of the patient 102, system parameters, and/or environmental parameters, all of which can be called patient data. In an example, the monitoring device 208 may include or be coupled to one or more sensors to sense the patient data.

The physiological parameters of the patient 102, for example, and without limitation, include data related to one or more physiological parameters, also referred to as physiological parameters data, which can assist the WMD 130 in detecting whether or not the patient 102 needs a shock or any other intervention or assistance. The physiological parameters data may also, in an example, include medical history of the patient 102, event history, and the like. The physiological parameters data further includes ECG, blood oxygen level, blood flow, blood pressure, blood perfusion, pulsatile change in light transmission or reflection properties of perfused tissue, heart sounds, heart wall motion, breathing sounds, and the pulse of the patient 102.

Accordingly, the monitoring device 208 includes one or more sensors configured to acquire physiological signals of the patient 102. In some embodiments, the one or more sensors or transducers may include the one or more ECG electrodes 106-112 to detect or obtain the ECG signals, a perfusion sensor, a pulse oximeter, a device for detecting blood flow, for example, a Doppler device, and the like. In some embodiments, the one or more sensors include a sensor for detecting blood pressure, for example, a cuff, an optical sensor, illumination detectors, and sensors perhaps working together with light sources for detecting color change in tissue. In some embodiments, the one or more sensors include a motion sensor, a device that can detect heart wall movement, a sound sensor, a device with a microphone, a SpO2 sensor, and the like. In view of the foregoing, it will be appreciated that such sensors can help detect the pulse of the patient 102, and can therefore also be called pulse detection sensors, pulse sensors, and pulse rate sensors. In addition, a person skilled in the art may implement other ways of performing pulse detection.

In some embodiments, the monitoring device 208 and/or the processor 222 may detect a trend in the monitored physiological parameters data of the patient 102. The trend may be detected by comparing values of parameters at different times over short and/or long terms. The physiological parameters, whose detected trends may help a cardiac rehabilitation program, include a) cardiac function, for example, ejection fraction, stroke volume, cardiac output, and the like; b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above; f) respiratory function, respiratory rate, and the like; g) motion, level of activity; and the like.

The detected trend may be stored and/or reported to a physician via one or more wired or wireless communication links, along with a warning to the physician monitoring progress or health status of the patient 102, if warranted. The reported trends provide clarity and updated information corresponding to the patient 102, to the physician. The physician may gauge if a condition is either not improving or deteriorating based on the reported trends.

The state parameters include recorded aspects of the patient 102, such as motion, posture, whether the patient 102 has spoken or communicated with a physician recently along with what has been spoken, and the like. In an example, the state parameters further include a history of the state parameters. In an example, the monitoring device 208 may include a location sensor such as a Global Positioning System (GPS) location sensor. The location sensor may detect the location of the patient 102, and speed of the patient 102 can be detected as a rate of change of location over time.

In some embodiments, the monitoring device 208 may include motion detectors that can be configured to detect a motion event and output a motion signal indicative of motion of the motion detector, and thus motion of the patient 102. The state parameters can assist in narrowing down the determination of whether Sudden Cardiac Arrest (SCA) is indeed occurring. In some embodiments, the WMD 130 includes a motion detector. The motion detector can be made in many ways as is known in the art, for example by using an accelerometer. The motion event can be defined as convenient, for example, a change in motion from a baseline motion or rest, and the like. In response to the detected motion event, the motion detector may render or generate a motion detection input that may be received by a subsequent device or functionality. In some embodiments, the WMS 100 may include a motion detector coupled to the WMD 130.

The system parameters may include system identification, battery status, system date and time, reports of self-testing, records of data entered, records of episodes and interventions, and the like. The environmental parameters may include ambient temperature and pressure. Moreover, a humidity sensor may provide information as to whether or not it is likely raining. The detected location of the patient 102 may also be considered as one of the environmental parameters. The location of the patient 102 may be presumed if the monitoring device 208 includes the GPS location sensor as mentioned above, and if the patient 102 is wearing the WMS 100.

The WMD 130 also includes the measurement circuit 210 that may be communicatively coupled to the monitoring device 208 and the one or more sensors/transducers. The measurement circuit 210 senses one or more electrical physiological signals of the patient 102 from the sensor port 204. In an embodiment, if the WMD 130 lacks the sensor port 204, the measurement circuit 210 may, in an example, obtain physiological signals through the electrical nodes 236, 238 instead, when the defibrillation electrodes 116, 118 are attached to the patient 102. The input to the measurement circuit 210 through the electrical nodes 236 and 238 is the ECG signal that reflects the ECG measurement. The patient data, in an example, is the ECG signal that may be sensed as a voltage difference between the defibrillation electrodes 116, 118. In addition, the patient parameter may be an impedance, which can be sensed between the defibrillation electrodes 116, 118 or between the connections of the sensor port 204 considered pairwise.

Sensing the impedance may be useful for detecting, among other things, whether the defibrillation electrodes 116, 118 and/or the ECG electrodes 106-112 are not making good electrical contact with the body of the patient 102. The physiological signals of the patient 102 may be sensed when available. The measurement circuit 210 can render or generate information about the physiological signals of the patient 102 as inputs, data, other signals, and the like. As such, the measurement circuit 210 may be configured to render a patient input responsive to the patient parameters sensed by a sensor. In some embodiments, the measurement circuit 210 may be configured to render the patient input, such as values of the ECG signal, responsive to the

ECG signal sensed by the ECG electrodes 106-112. Although the information rendered by the measurement circuit 210 is output from it, the information may be called an input because the information is received as an input by a subsequent device or functionality.

The WMD 130 further includes the memory 212, which is communicatively coupled with the processor 222. The memory 212 may be implemented in a number of ways, such as but not limited to, volatile memories, Non-Volatile Memories (NVM), Read-Only Memories (ROM), Random Access Memories (RAM), magnetic disk storage media, optical storage media, smart cards, flash memory devices, any combination thereof, and the like. The memory 212 is, thus, a non-transitory storage medium that may include programs for the processor 222, which the processor 222 may be able to read and execute. More particularly, the programs may include sets of instructions in the form of code, which the processor 222 may be able to execute upon reading. The programs may also include other information such as configuration data, profiles, scheduling, and the like that may be acted upon by the instructions. The execution is performed by physical manipulations of physical quantities and may result in functions, operations, processes, acts, actions, and/or methods to be performed. In some embodiments, the processor 222 is configured to cause other devices, components, or blocks to perform functions, operations, processes, acts, actions, and/or methods mentioned above.

The non-transitory computer-readable storage medium is encoded or configured to store computer program instructions defined by one or more modules. The non-transitory computer-readable medium is encoded with instructions for filtering noise from one or more ECG signals which when executed by a computing device, such as the processor 222, cause the computing device to perform operations for filtering noise from the one or more ECG signals. In some embodiments, instances of the software may be referred to as a “module” and by other similar terms. However, the term “module” used in the context of disclosure is intended to be broad and may include hardware, software, distributed components, remote components, for example, cloud computing, and the like. Further, a module includes a set of instructions to offer or fulfill a particular functionality. The memory 212 includes a noise filter module 214, a dominant beat detection (DBD) module 216, an R-R interval module 218, and a detector module 220.

The noise filter module 214 receives the one or more ECG signals from the one or more ECG electrodes 106-112. In some embodiments, the received ECG signals may be down-sampled, which allows the noise filter module 214 to perform one or more operations related to reduction or elimination of noise from the received ECG signals. The noise filter module 214 is configured to sample the received ECG signals at a predetermined or predefined frequency. In some embodiments, the received ECG signals may be sampled at a predefined frequency of about 500 Hz. In other embodiments, the received ECG signals may be sampled at different sampling rates other than 500 Hz.

The noise filter module 214 determines if the received or the sampled ECG signals are noisy. In some embodiments, the noise filter module 214 determines if any of the ECG leads are off, and based on the determination, the noise filter module 214 identifies the corresponding ECG signal as noisy. In some embodiments, the noise filter module 214 determines if a slope of consecutive samples across segments of the ECG signals i.e., a magnitude of difference between a current and a previous sample, exceeds a first threshold. One or more samples of the ECG signal that exceed the first threshold are classified as noise. The first threshold is derived or obtained by considering clean ECG data with normal ECG signals, analyzing different ECG signals from a large set of ECG signals, and/or considering a value for the first threshold that is higher than a highest peak in the normal ECG signals. The first threshold, for example, is 2 mV/sample.

In some embodiments, the noise filter module 214 determines if an amplitude difference between a first sample and a last sample in a series of samples exceeds a second threshold. The series of samples may include at least ten samples where the first sample is a first of the ten samples and the last sample is a tenth of the ten samples. A value for the second threshold is determined by empirical analysis of noise data. One or more samples of the ECG signal that exceed the second threshold are considered noise. The second threshold, for example, is 5 mV. In some embodiments, the second threshold is greater than the first threshold. Further, in some embodiments, the ECG signals obtained when any lead corresponding to the ECG electrodes 106-112 is off are classified as noise. By using the first threshold, the noise filter module 214 may be capable of identifying segments or samples of noise that are shorter and include spikes. Further, by using the second threshold, the noise filter module 214 may be capable of identifying segments or samples that are longer compared to other segments of the ECG signals. The first threshold and the second threshold may be collectively referred to as noise threshold. The noise filter module 214 provides a blanking period if the slope of the consecutive samples exceeds the first threshold or if the amplitude difference of the series of samples exceeds the second threshold.

The samples of the ECG signals that are identified as noise along with samples that are present before or after the noise samples are blanked. The blanking of the noise samples prevents the detection of any QRS complexes that may lead to erroneous calculations related to the arrhythmia detection such as but not limited to AF detection. In some embodiments, a blanking period is about 700 ms before and after each noise sample. The blanking period replaces values of the ECG signals in the blanking period with placeholders such as Not a Number (NaN) values, for example, while keeping the other samples of ECG signals set in time or at their proper time location. In some embodiments, a NaN is a convenient placeholder because it takes the place of an ECG sample but does not have a numerical value. The blanking may be accomplished, for example, by replacing the noise samples with the NaN values, thereby removing noise and retaining other samples at respective time locations. The replacement of noise samples with NaN values is different from replacing noise samples with zeros. The value zero represents a legitimate ECG signal value and is different from deleting noise samples which may shift samples of the ECG signals, other than noise samples, in time. It shall be noted that placeholders other than NaN may be used in some embodiments to accomplish the same purpose without deviating from the scope of the present disclosure. For example, a specific numerical value such as 2,147,483,647 might be designated to represent a NaN. Alternatively, a second array or list may be kept to mark the locations of NaN values in an ECG array without limitation.

In some embodiments, the portion of the signal containing NaN may be expanded to include a portion of the signal in the vicinity of the noise detection. In an exemplary embodiment, the expansion may be implemented with a digital filter such as filter 214A. Filters 214A included in the noise filter module 214, such as a digital filter with a corresponding filter window, may be used on the ECG signals. The filter window is also referred to as a blanking window. The digital filter automatically replaces samples equivalent to length of the digital filter or the blanking window. Further, the digital filter also blanks samples around the samples equivalent to the length of the digital filter with the NaN values. As a result, the noise filter module 214 generates a primary signal with the NaN values. The blanking window may be expanded by a predetermined number of samples or time period to blank the noise samples of the ECG signals.

The noise filter module 214 may filter the generated primary signal with the NaN values by using one of the filters 214A, such as a bandpass filter that may include a high pass filter and a low pass filter. The bandpass filter stabilizes baseline and attenuates high frequency noise. In one embodiment, the bandpass filter has a cut-off frequency of about 8 Hz and 25 Hz. The bandpass filter, for example, may be a Finite Impulse Response (FIR) filter. In some embodiments, the noise filter module 214 may use one of the filters 214A, such as a bi-directional filter, to filter the generated primary signal with the NaN values. The bi-directional filter filters the generated primary signal with the NaN values in both directions by a predetermined number of samples based on length of the bi-directional filter.

By filtering the generated primary signal, a secondary signal, also referred to as the filtered ECG signal, is generated with the NaN values that spread beyond the NaN values in the generated primary signal. In some embodiments, a blanking window corresponding to the bi-directional filter may be expanded based on a certain number of samples or time period. The filters 214A, such as the bi-directional filter, in some embodiments, do not include a roll-off and have a flat unity gain across an entire frequency space that supports expandability of the length or the blanking window of the filters 214A. In some embodiments, the spreading of the blanking period is controlled by the length of the filters 214A.

In some embodiments, the noise filter module 214 employs filtering algorithms that return the NaN value if any of the samples in corresponding filter delay line are the NaN values. The number of “expanded” samples in the secondary signal is based on the order of at least one of the filters among the filters 214A. For example, in some embodiments, the length of the bidirectional filter is about 350 samples causing each of the samples with the NaN values to be expanded for about 350 samples. If the bi-directional filter is run in both directions, then a single sample with the NaN value is expanded to 350 samples before and after each noise sample, summing up to 700 samples.

For example, for a signal sampled at 500 Hz, the bi-directional filter may blank a total of 1.4 seconds of the signal. The blanking may help in avoiding situations or calculations due to the presence of noise artifacts before or after a point where the ECG signal reaches the noise threshold and is first detected. In some embodiments, the noise filter module 214 additionally blanks the non-blanked samples of the ECG signals having a duration of less than 30 seconds between two blanking periods. If a blanking period is followed by 10 seconds of a valid non-blanked segment or samples of the ECG signal and then followed by another blanking period, then 10 seconds of the valid non-blanked segment would be additionally blanked.

In some embodiments, the blanking period includes about 250 samples to about 450 samples before and after the consecutive samples that exceed the first threshold or the series of samples that exceed the second threshold. Therefore, the additional blanking removes short, stranded ECG periods from littering the ECG signal which may not support meaningful analysis of the ECG signals; thereby saving resources such as power, memory, and the like, of the WMD 130. The additional blanking also reduces the possibility of probable undetected noise segments surrounding the identified noise segments to affect the determination of the arrhythmia (e.g., AF) by the detector module 220. In some embodiments, the NaN values may be added using computational platforms or software, such as but not limited to MATLAB®, or computer languages like python or C language. In some embodiments, the blanking of the noise segments of the ECG signal may be performed using a separate array that labels each sample as “blanked” or “not blanked.”

Further, upon blanking the noise segments for each channel, the noise filter module 214 determines if the filtered ECG signal includes about 25 percent or more of noise samples or the NaN values. The noise filter module 214 may also determine if the ECG signal has less than three minutes of contiguous data in the filtered ECG signal. Upon determination, the noise filter module 214 identifies and labels corresponding channels as noisy and deletes or ignores the ECG signals from the noisy channels for further calculation or consideration. The noise filter module 214 then deletes the corresponding received ECG signals from the WMD 130. If the WMD 130 determines that, for example, less than two channels remain, upon labeling the channels as noisy, then the WMD 130 does not proceed with the arrhythmia or anomaly detection (e.g., AF) with the corresponding ECG signals. The noise filter module 214 then provides the filtered ECG signals to the DBD module 216.

The DBD module 216 is configured to perform one or more operations for identifying dominant beats from input received from the noise filter module 214 within a predetermined time frame. The DBD module 216, after receiving the input, continually or periodically performs the one or more operations on the received inputs during the wear-time of the WMD 130 or WMS 100. The input to the DBD module 216 may include ECG signals from the identified multiple channels that are not labeled as noisy. The DBD module 216 may utilize at least a portion of the ECG signals from the identified multiple channels to identify one or more dominant supraventricular beats, also referred to as dominant beats, in the portion. The DBD module 216 includes a QRS detector (not shown) to identify QRS complexes or beats in the received input. In some embodiments, the QRS detector in the DBD module 216 is similar to a QRS detector used in the ASSURER Wearable defibrillator. In some embodiments, the WMD 130 includes another QRS detector as a gatekeeper QRS detector along with the QRS detector in the DBD module 216.

In some embodiments, the DBD module 216 finds peaks in the received input. Specifically, the DBD module 216 may consider one or more parameters to identify the beats by finding the peaks in the QRS complexes of the received input. The one or more parameters include one or more of a minimum distance between peaks value, a minimum peak width value, a maximum peak width value, a minimum amplitude value, and the like. In some embodiments, the minimum distance between peaks value is about 200 ms, the minimum peak width value is about 10 ms, the maximum peak width value is about 500 ms, and the minimum amplitude value is about 0.2*max signal value for identifying the peaks. However, it will be apparent to a person with ordinary skill in the art that values for each of these one or more parameters are exemplary and may vary based on different inputs.

Upon detecting the peaks, the DBD module 216 divides the input from each of the identified multiple channels into four segments. The DBD module 216 determines an average R-R interval for each four second segment and excludes segments with R-R intervals greater than two seconds for each of the identified channels. Further, the DBD module 216 determines an average of a whole segment with the samples of the ECG signal and determines segment agreements between the identified channels. If the segment agreement is within an agreement threshold, such as 40 samples between one or more pairs of the identified channels, the DBD module 216 considers the ECG signals from the channels that satisfy the agreement threshold for further operations. The segment agreement between a pair of channels is defined as a number of four second segments that agree within 40 samples. Specifically, two channels of the identified multiple channels with the maximum segment agreement are determined and considered for further analysis.

Further, the DBD module 216 determines if, for each detection of a QRS complex in one channel of the pair of identified channels, another channel of the pair of identified channels has detection of QRS complex within a predetermined time, for example, 150 ms. If the QRS complex fails to be detected or present within the predetermined time in another channel, then the detected QRS complex is discarded. The detected QRS complexes from the channels with sufficient segment agreement may be utilized for further operations. Subsequently, the DBD module 216 identifies beats corresponding to the detected QRS complexes.

On identifying a beat, which may be a first beat, the DBD module 216 saves shape of the first beat as a first template in a template table and records a count corresponding to the first template. The count corresponding to the first template is set as 1, initially, in the template table. On identifying a next beat, the DBD module 216 compares the template corresponding to the first beat in the template table with a shape of the next beat. If the shape of the next beat matches with the first template, the count corresponding to the first template is updated or incremented as 2 in the template table. The shape of the first beat and the next beat are then averaged, which includes superimposing the next beat on the first beat and subsequently stored as a single template i.e., the first template. However, if the shape of the next beat does not match the first template or is unique, the shape of the next beat is stored as a second template in the template table.

Further, each consecutive beat is compared with stored and averaged templates, and upon determining a match, the DBD module 216 updates the count of corresponding templates in the template table. However, in a situation where the DBD module 216 determines a mismatch between the stored templates of the previous beats and the shape of consecutive beats, the shapes of the mismatched consecutive beats are stored as templates. Further, a count may be associated with the templates of the consecutive beats and set as 1, initially. The DBD module 216 then identifies the template with highest count and sets the beats that match the identified template as dominant beats.

The DBD module 216 compares the templates with the shape of the consecutive beats using cross-correlation and amplitude of the beats. The cross-correlation considers beats with same shape or morphology even though respective amplitudes differ. The beat may be determined to be matched with the stored and/or averaged template if one or more of maximum normalized cross-correlation is greater than 0.5, difference in peak amplitude absolute value is less than 0.25 mV, and/or a ratio of beat amplitude/template amplitude is greater than 0.5. The identified peaks are used by the R-R interval module 218 to measure the R-R interval between consecutive beats that are detected as the dominant beats. The measurements of the R-R interval are provided to the detector module 220.

By filtering the noise, replacing the noise segments with the NaN values, and further filtering the generated ECG signal with the NaN values using the bi-directional filter as one of the filters 214A, the DBD module 216 locates the dominant beats accurately. Further, the R-R interval module 218 considers only the located dominant beats for determining the R-R intervals. The filtering of the noise samples avoids a situation of miscalculating the R-R intervals that would affect the Heart Rate (HR) variability determination and the arrhythmia detection by the detector module 220. In some embodiments, the R-R interval module 218 is present in the DBD module 216.

The detector module 220 is configured to receive output data from the R-R interval module 218 to determine one or more detection metrics or digital biomarkers. Further, based on the received output data from the R-R interval module 218, the detector module 220 determines R-R interval variability that may support indicating presence of arrhythmia, such as AF. The detector module 220 utilizes R-R intervals between dominant beats that increases accuracy of the arrhythmia detection like AF since the detector module 220 avoids inclusion of the noise, non-supraventricular beats, or incorrectly detected R-wave detections. In some embodiments, the detection metrics for AF, as an example, include at least one of Shannon entropy, origin count, irregularity, stepping, dispersion, and the like. In some embodiments, the detection metrics include determining RR interval variation comprising metrics related to randomness or irregularity of the R-R intervals. The detector module 220 employs at least one method corresponding to machine learning that utilizes the one or more detection metrics for determining presence or absence of the AF. The method corresponding to the machine learning may be at least one of decision tree method or random forest method that uses the one or more detection metrics for determining the presence or absence of the AF. The methods corresponding to machine learning may be any suitable method that utilizes R-R interval or R-R variability as an input parameter. The method may be trained based on different databases, such as public databases, proprietary data, and the like. For example, the public database may include Long Term AF, Massachusetts Institute of Technology (MIT)-Beth Israel Hospital (BIH)-AF database, MIT-BIH-Arrhythmia database, MIT-BIH-Supraventricular Arrhythmia (SVT) Database, Paroxysmal Atrial Fibrillation (PAF) Prediction Challenge database, American Heart Association (AHA) database, Telemetric and Holter ECG Warehouse (THEW) AF Cardioversion database, and the like. The choice of method may be dependent on explainability and accuracy of the corresponding method.

In some embodiments, the detector module 220 further determines AF burden corresponding to the patient 102. The detector module 220 calculates percentage of time a patient is in the AF. For example, if the patient 102 spends one hour in the AF during a day, the patient 102 may have an AF burden of about 4.2%, where the AF burden is calculated as 1/24*100. However, if the signal has been blanked by the noise filter module 214 for about four hours during the day, then the AF burden may be about 5%, where the AF burden is calculated as 1/20*100. The calculated AF burden may be communicated to a physician or medical personnel corresponding to the patient 102. Based on the AF burden along with the detection of the AF and the HR variability, the physician or medical personnel may determine the next course of action.

The WMD 130 also includes the processor 222 which may be implemented in different ways in various embodiments. The different ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination thereof, and the like.

The processor 222 may have access to a non-transitory storage medium, such as the memory 212 that, in some embodiments, is a non-volatile component for storage of machine-readable and machine-executable instructions for filtering the noise and determining the AF. A set of such instructions can also be called a program. The instructions, which may also be referred to as “software,” generally provide functionality by performing acts, operations, and/or methods as may be disclosed herein or understood by one skilled in the art in view of the disclosed embodiments. The processor 222 may perform one or more operations on the ECG signals using the one or more modules of the memory 212 in different domains, such as time domain, frequency domain, and the like.

The WMD 130 includes the communication module 224 for establishing the one or more wired or wireless communication links with other devices of other entities, such as a remote assistance center, Emergency Medical Services (EMS), and the like. The communication links may be used to transfer data and commands. The data may be patient data, event information, therapy attempted, Cardiopulmonary resuscitation (CPR) performance, system data, environmental data, and so on. For example, the communication module 224 may wirelessly transmit data such as heart rate, respiratory rate, and other vital signs data daily to a server accessible over the internet, for instance as described in U.S. Pat. Publication No. 2014/0043149 A1.

The physician of the patient 102 may directly analyze the communicated data or the communicated data may also be analyzed automatically by algorithms designed to detect a developing illness and then notify medical personnel via text, email, phone, and the like. The communication module 224 may also include interconnected sub-components which may be deemed necessary by a person skilled in the art, for example, but not limited to, an antenna, portions of the processor 222, supporting electronics, an outlet for a telephone or a network cable, and the like.

In some embodiments, the WMD 130 may then transmit the data to a remote location, for example, a KESTRA™ CARESTATION remote data portal, or to a doctor of the patient 102. The data can then be viewed, for example, by a medical professional in treating or monitoring the patient 102.

The therapy unit 226 of the WMD 130 includes a power source 228, an energy output module 230, a discharge circuit 232, and the defibrillation port 234 coupled to the defibrillation electrodes 116, 118. To enable portability of the WMD 130, the power source 228, in some embodiments, includes a battery. The battery, for example, is a battery pack, which may either be rechargeable, non-rechargeable, or a combination of both. An embodiment of the power source 228 may include an alternate current (AC) power override, for where AC power will be available, an energy-storing capacitor, and so on. Appropriate components may be included to provide for charging or replacing the power source 228. In some embodiments, the power source 228 is controlled and/or monitored by the processor 222.

The WMD 130 further may include the energy output module 230 that may be coupled to the support structure 104 of the WMS 100, for example, either directly or via the defibrillation electrodes 116, 118, and respective leads. The energy output module 230 temporarily stores electrical energy as an electrical charge, when preparing for discharge of the electrical charge to administer the shock to the patient 102 and may be charged from the power source 228 to the desired amount of energy. The energy output module 230 includes a capacitor C1, which may be a single capacitor or a system of capacitors, and the like. A decision to deliver a shock may be made responsive to the shock criterion being met. At least some or all of the electrical charge stored in the energy output module 230 may be discharged through the patient 102 while the support structure 104 is worn by the patient 102, to deliver the shock to the patient 102, based on the shock criterion.

For causing the discharge, the WMD 130 includes the discharge circuit 232. When the decision is to deliver the shock, the processor 222 may be configured to control the discharge circuit 232 to discharge through the patient 102 at least some of or all of the electrical charge stored in the energy output module 230. The discharging may be performed to the electrical nodes 236, 238, and then to the defibrillation electrodes 116, 118, causing the shock to be delivered to the patient 102. The discharge circuit 232, for example, includes one or more switches S1. The switches S1 may be made or arranged in a number of ways, such as by an H-bridge, and the like. The discharge circuit 232 may also be controlled via the processor 222 and/or the user interface 206. In some embodiments, the therapy unit 226 may be an optional block in the WMD 130. In some embodiments, the WMD 130 may be utilized for filtering the noise corresponding to the ECG signals and detecting the arrhythmia like AF without providing therapy by administering the shock.

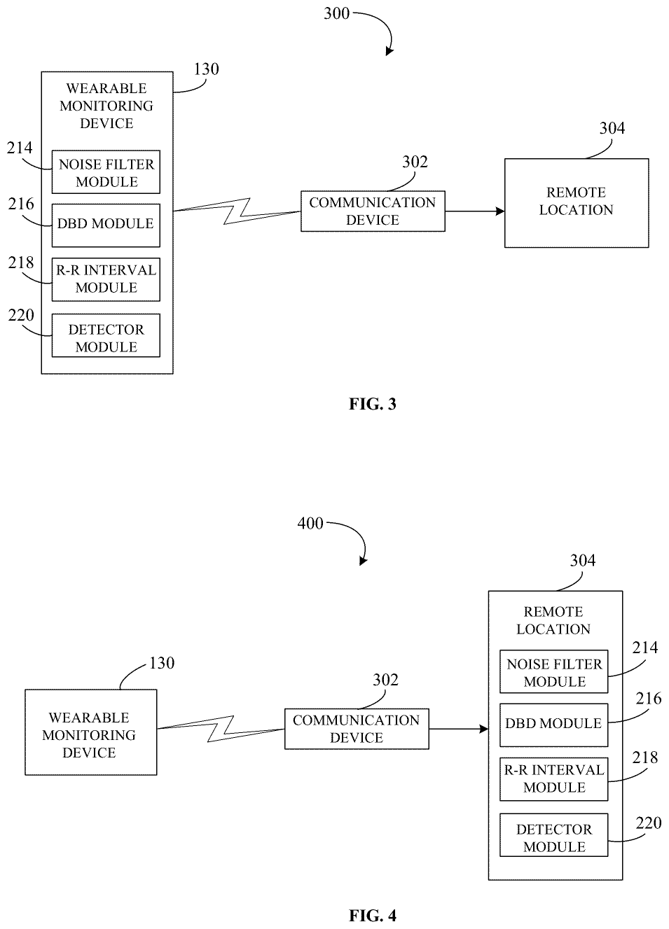

FIGS. 3 and 4 illustrate block diagrams 300 and 400 that depict communication between the WMD 130 and a remote location 304, according to some embodiments of the present disclosure. In some embodiments, the WMD 130 may be included in an ASSURER wearable defibrillator system. The remote location 304 is, for example, a server, a cloud-based web service, a device, or a unique communication number of a family member of the patient 102, a physician of the patient 102, any other medical professional, and the like. In some embodiments, the remote location 304 is similar to the remote location disclosed in FIG. 2. The remote location 304 is a cloud-based portal, for example, KESTRA™ CARESTATION remote data platform. In some embodiments, the remote location 304 may be implemented using a web service such as, for example, Amazon Web Services® (AWS®) or Microsoft Azure®.

Block diagram 300 illustrates an embodiment where the WMD 130 includes the one or more modules, as discussed in FIG. 2, that aid in detecting or determining the arrhythmia (e.g., AF). Information regarding the determination of the arrhythmia is provided to the remote location 304 through a communication device 302 that is communicatively coupled to the WMD 130 and the remote location 304. The information or results from the WMD 130 can then be viewed, for example, by a medical professional in treating or monitoring the patient 102.

In some embodiments, the WMD 130 communicates with the communication device 302 through a wired or wireless medium. In some embodiments, the WMD 130 may transmit the patient data and data corresponding to the WMD 130 to the remote location 304. The WMD 130 may also transmit outputs from each of the one or more modules to the remote location 304, through the communication device 302. A medical professional corresponding to the patient 102 may be at the remote location 304 and view the patient data and data corresponding to the WMD 130 for treating or monitoring the patient 102.

Block diagram 400 illustrates an embodiment where the remote location 304 includes the one or more modules such as the noise filter module 214, the DBD module 216, the R-R interval module 218, and the detector module 220. The WMD 130 transmits the patient data, such as the ECG signals, to the remote location 304 via the communication device 302. The remote location 304 receives the patient data and performs operations on the received patient data to determine the AF. In some embodiments, the medical professional may view the determination and instruct the WMD 130, through the communication device 302, to provide therapy to the patient 102 using the therapy unit 226. In some embodiments, the communication device 302 is integrated into the WMD 130 rather than being a separate device.

In some embodiments, the one or more modules of the WMD 130 may be distributed between the WMD 130 and the remote location 304. For example, the noise filter module 214 may be present in the WMD 130 and the other one or more modules such as the DBD module 216, the R-R interval module 218, and the detector module 220 may be present in the remote location 304. The WMD 130 may provide output of the noise filter module 214 to the remote location 304 that includes the other one or more modules. The remote location 304 performs operations on the received output from the noise filter module 214 and determines the presence of the AF. The medical professional corresponding to the patient 102 may review output of the one or more modules, at the remote location 304.

Although two specific configurations illustrating distribution or split of modules and their corresponding functionalities are depicted in FIGS. 3 and 4, however, it will be apparent to a person with ordinary skill in the art that other distributions or configurations are possible to detect the arrhythmia like AF without deviating from the scope of the present disclosure.

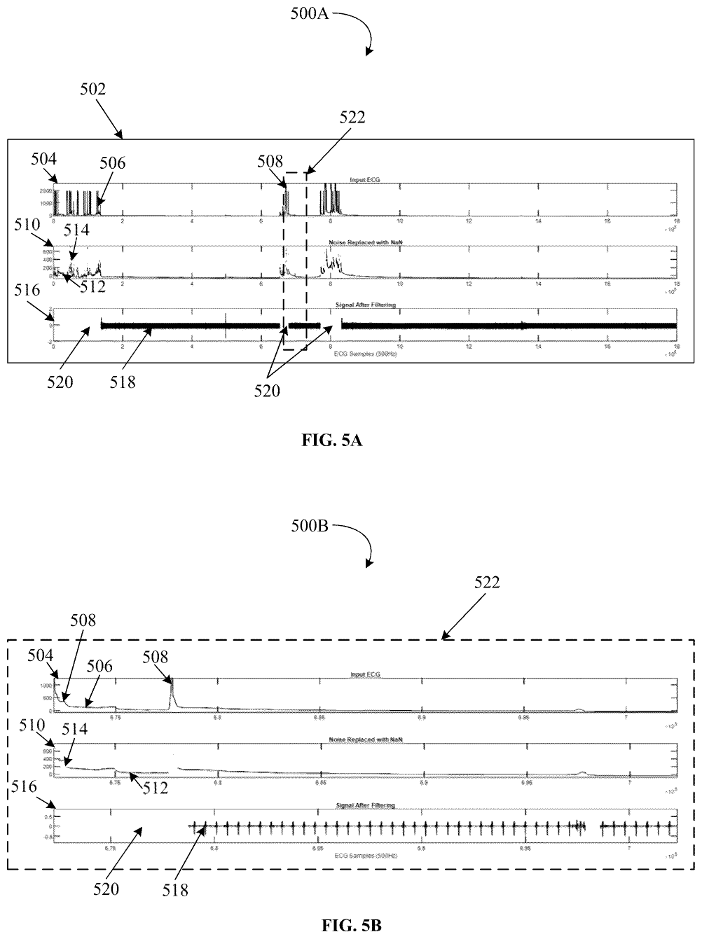

FIGS. 5A and 5B illustrate graphs 500A and 500B depicting the input to and output from the noise filter module 214, according to an embodiment of the present disclosure. FIGS. 5A and 5B are described in conjunction with the previous figures. The graph 500A includes data 502 captured for a time period of one hour from the noise filter module 214. The data 502 includes a first waveform 504 of an ECG signal 506 sensed by the ECG electrodes 106-112, in some embodiments, sampled at, for example, 500 Hz. The ECG signals 506 include one or more ECG signals from the multiple channels of the WMS 100.

The first waveform 504 corresponds to the ECG signal 506 which is corrupted by noise or includes noise artifacts that are high amplitude portions of the ECG signal 506, such as a noise sample 508. The ECG signal 506 also includes QRS complexes that may be about, for example, 1 millivolt (mV), and the noise sample 508, on the other hand, may be about a few thousand millivolt, for example, 2000 mV (2V) or more. The noise threshold (not shown) may be used to identify the noise sample 508 in the first waveform 504 based on which the first waveform 504 is processed. In some embodiments, the noise sample 508 may have amplitudes of about 2000 mV or above. The first waveform 504 is processed based on at least the noise threshold to obtain a second waveform 510. The second waveform 510 is similar to the generated primary signal as discussed previously in the description of FIG. 2. The noise filter module 214 identifies the noise sample 508 based on the noise threshold and provides a processed ECG signal 512 and replaces the noise sample 508 with NaN values 514, in the processed ECG signal 512. As a result, peak amplitude of the processed ECG signal 512 is about 600 μV which is significantly lesser than the peak amplitude of the ECG signal 506.

The noise filter module 214 provides a third waveform 516 as an output to subsequent modules, for example, the DBD module 216. The third waveform 510 is similar to the secondary signal as discussed previously in the description of FIG. 2. The noise filter module 214 further processes the processed ECG signal 512 with the NaN values 514 of the second waveform 510 by blanking portions of the ECG signal 512 that include the NaN values 514. The ECG signal 512 is filtered using the bi-directional filter as one of the filters 214A included in the noise filter module 214. The output of the noise filter module 214 is the third waveform 516 that includes a filtered ECG signal 518 with blanked period 520, also referred to as blanking periods 520. The filtered ECG signal 518 has peak amplitudes of about ±0.5 μV with respect to typical ECG voltages.

The graph 500A includes an observation window 522 overlaid on the first waveform 504, the second waveform 510, and the third waveform 516. The graph 500B illustrates magnified or enlarged regions of the first waveform 504, the second waveform 510, and the third waveform 516 within the observation window 522. The graph 500B provides a detailed view of at least the third waveform 516 illustrating the filtered ECG signals with amplitudes of about ±0.5 μV.

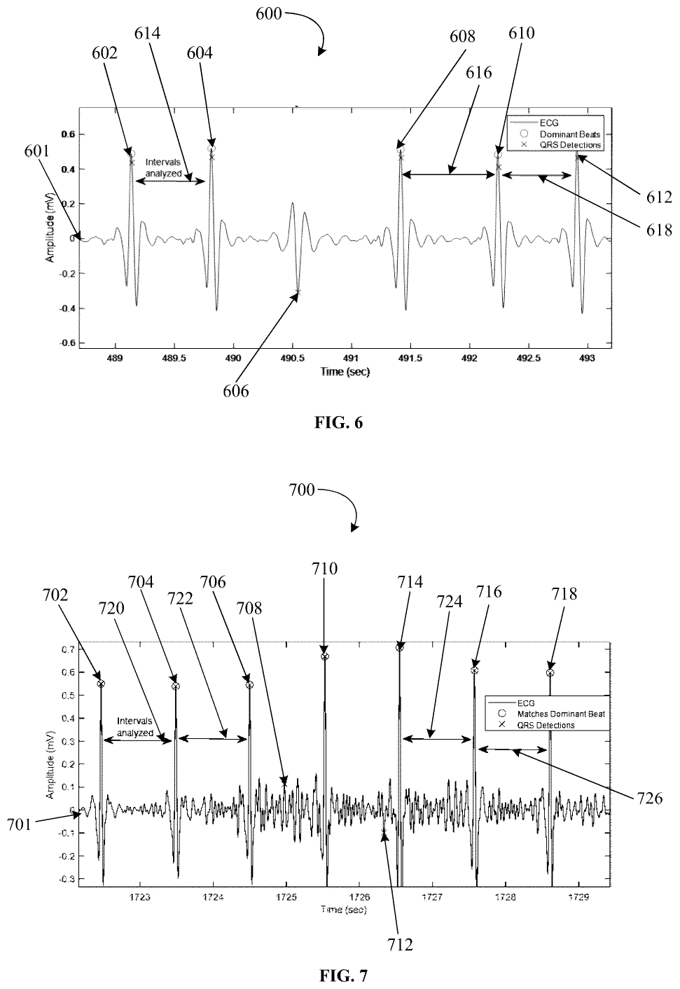

FIG. 6 illustrates a graph 600 that depicts identification of dominant and non-dominant beats in a clean ECG signal 601 that is noise free and R-R interval between consecutive dominant beats on a single channel of the plurality of channels, according to an embodiment of the present disclosure. As described previously, the output of the noise filter module 214 is provided to the DBD module 216 to identify the dominant beats. The QRS detector of the DBD module 216 initially identifies beats 602, 604, 606, 608, 610, and 612 as R waves of the QRS complexes. The identified beats 602, 604, 606, 608, 610, and 612 are indicated with a cross on respective R-waves on the graph 600. Further, the dominant beat identifier of the DBD module 216 identifies beats 602, 604, 608, 610, and 612 as the dominant beats. The dominant beats 602, 604, 608, 610, and 612 are indicated with a circle on the respective R-waves on the graph 600. The DBD module 216 considers the beat 606, which is of a shape different from the beats 602, 604, 608, 610, and 612, as noise and to consider the beat 606 as a non-dominant beat. The heart rate for a person with regular heart function is steady, however, the presence of the non-dominant beat 606 may be a result of Premature Ventricular Contractions (PVCs) that may be random. By ignoring the non-dominant beat 606, a situation is eliminated where the HR variability may seem different from actual HR variability.

The R-R interval module 218, upon receiving the identified dominant beats, determines the R-R interval between the identified consecutive dominant beats. In some embodiments, the identified consecutive dominant beats are similar to the dominant beats as disclosed in the description of FIG. 2. The R-R interval module 218 identifies an interval 614 between the identified consecutive dominant beats 602 and 604, an interval 616 between the identified consecutive dominant beats 608 and 610, and an interval 618 between the identified consecutive dominant beats 610 and 618. The R-R interval module 218 does not determine the interval between the identified dominant beats 604 and 608 due to the presence of the non-dominant beat 606 in between.

FIG. 7 illustrates a graph 700 that depicts identification of dominant and non-dominant beats in an ECG signal 701 that is noisy and determination of R-R intervals between consecutive dominant beats, according to an embodiment of the present disclosure. As described previously, the output of the noise filter module 214 is provided to the DBD module 216 for identifying the dominant beats. The QRS detector initially identifies beats 702, 704, 706, 708, 710, 712, 714, 716, and 718 as R waves of the QRS complexes. The identified beats 702, 704, 706, 708, 710, 712, 714, 716, and 718 are indicated with a cross on respective R-waves on the graph 700. Further, the DBD module 216 identifies beats 702, 704, 706, 710, 714, 716, and 718 as the dominant beats. The DBD module 216 may determine that the identified beats 708 and 712 did not reach the noise threshold corresponding to the noise filter module 214 and label the beats 708 and 712 as non-dominant beats. The dominant beats 702, 704, 706, 710, 714, 716, and 718 are indicated with a circle on the respective R-waves on the graph 700. The DBD module 216 may consider the beats 708 and 712, which are of a shape different from the beats 702, 704, 706, 710, 714, 716, and 718, as the noise. The non-dominant beats 708 and 712 may be a result of the PVC, as discussed in the description of FIG. 6.

The R-R interval module 218, upon receiving the identified dominant beats, determines the R-R interval between the identified consecutive dominant beats. The R-R interval module 218 identifies intervals 720, 722, 724, and 726 between the identified consecutive dominant beats 702 and 704, 704 and 706, 714 and 716, and 716 and 718, respectively. The R-R interval module 218 does not determine the interval between the identified dominant beats 706 and 710 due to the presence of the non-dominant beat 708 in between, and the interval between the identified dominant beats 710 and 714 due to the presence of the non-dominant beat 710 in between. By considering the intervals solely between the consecutive dominant beats 702, 704, 706, 714, 716, and 718 with no beat classified or labeled as noise, the R-R interval module 218 determines the true or actual heart rate of the patient 102.

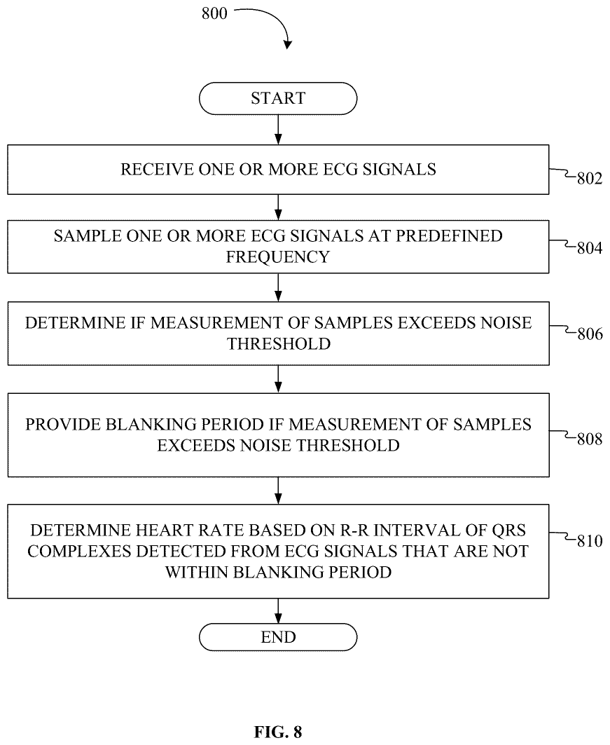

FIG. 8 illustrates an example method 800 for filtering noise from the one or more ECG signals 506 using the WMD 130, according to an embodiment of the present disclosure. FIG. 8 is described in conjunction with the previous figures. Although the example method 800 depicts a particular sequence of operations, the sequence may be altered without departing from the scope of the present disclosure. For example, some of the operations depicted may be performed in parallel or in a different sequence that does not materially affect the function of the method 800. In other examples, different components of an example device or system that implements the method 800 may perform functions at substantially the same time or in a specific sequence.

The method 800 begins, at block 802, by detecting or receiving one or more ECG signals 506 using the plurality of ECG electrodes 106-112. In some embodiments, the WMD 130 may sense the one or more ECG signals 506 using the plurality of ECG electrodes 106-112 attached to or positioned on the patient 102.

Upon receiving the one or more ECG signals 506, the method 800, at block 804, includes sampling the one or more ECG signals 506 at a predefined frequency using the noise filter module 214. In some embodiments, the predefined frequency is about 500 Hz. The method 800, at block 806, further includes determining if a measurement of samples exceeds a noise threshold (not shown). The measurement includes a first measurement which is an absolute value of an amplitude difference between the consecutive samples, and a second measurement is an absolute value of an amplitude difference between a first sample and a last sample in a series of sample. Further, the noise threshold includes a first threshold which is about 2 mV/sample, and a second threshold which is about 5 mV. The slope of the measurement may be considered as a first derivative of an ECG signal of the one or more ECG signals 506. If the slope or absolute value of the ECG signal 506 is greater than the first threshold, then the noise filter module 214 determines corresponding portion of the ECG signal 506 as noise. In some embodiments, the first threshold is about 2 m V/sample.

The method 800 further includes determining if an amplitude difference between a first sample and a last sample in a series of samples exceeds a second threshold (not shown), using the noise filter module 214. In some embodiments, the first sample and the last sample in the series of samples may be 10 samples apart. In some embodiments, the second threshold is about 5 m V between the first sample and the last sample in the series of samples. In some embodiments, the second threshold is greater than the first threshold.

The method 800, at block 808, further includes providing the blanking period 520 if the measurement of samples exceeds the noise threshold, using the noise filter module 214. The noise filter module 214 then replaces such consecutive samples or the series of samples with the NaN values 514. Further, the bi-directional filter as one of the filters 214A is configured to blank samples before and after the consecutive samples that exceed the first threshold or the series of samples that exceed the second threshold for implementing the blanking period 520. The blanking period 520 replaces values of ECG signals of the one or more ECG signals 506 in the blanking period 520 with the NaN values 514, thereby keeping the ECG signals of the one or more ECG signals 506 set in time. In some embodiments, the blanking period 520 further comprises about 250 samples to about 450 samples before and after the consecutive samples that exceed the first threshold or the series of samples that exceed the second threshold.

The method 800 further includes filtering the samples with a filter of one of the filters 214A and, at block 810, determining the heart rate of the patient 102 based on an R-R interval of the QRS complexes detected from the ECG signals of the one or more ECG signals 506 that are not within the blanking period 520.

The WMD 130, along with the corresponding method 800, is utilized for filtering noise from the ECG signals by blanking noise samples of the ECG signals obtained from a patient through multiple channels. The WMD 130 further identifies and selects channels that may be noise free or include noise, which is less than a predefined noise threshold, and utilizes the ECG signal(s) from the selected channels for detection of beats of QRS complexes corresponding to the ECG signals. One or more of the detected beats are further identified as dominant beats based on uniqueness of shape of the beats and corresponding count. The WMD 130 calculates R-R interval between the dominant beats and HR variability. The WMD 130 further detects presence or absence of the arrhythmia (e.g., AF). By filtering the noise from the ECG signals, selecting relevant channels, and detecting dominant beats improves accuracy of detection of the arrhythmia. Further, by blanking the noise samples, the WMD 130 reduces the computation requirement for accurately determining the arrhythmia. Further, the WMD 130, by using the previously mentioned one or more modules, improves specificity, sensitivity, and positive predictivity corresponding to the detection of the arrhythmia (e.g., AF) compared with conventional arrhythmia detectors.

It shall be noted that some of the embodiments described herein focus on AF detection as an application of noise detection and blanking. However, without deviating from the scope of the present disclosure, AF detection is one of the many examples of the analysis that could be performed. Any device that performs a rhythm analysis may avoid incorrect rhythm analysis results by blanking periods of noise without limitation.

Thus, in the embodiments discussed herein, noise filtering of ECG signals is crucial to obtain clean ECG signals for better diagnosis and prognosis. As a non-limiting example, analysis of clean ECG signals may play an important role for morphology/template-based algorithms and other rhythm analysis algorithms.

Other embodiments include combinations and sub-combinations of features described or shown in the drawings herein, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A medical system, comprising:
a plurality of ECG electrodes to sense one or more ECG signals of a patient; and
a processor in communication with the plurality of ECG electrodes, the processor configured to:
receive the one or more ECG signals;
sample the one or more ECG signals at a predefined frequency;
determine if a measurement of samples exceeds a noise threshold; and
provide a blanking period if the measurement of the samples exceeds the noise threshold;
wherein the blanking period replaces the samples in the blanking period with a not a number (NaN) value, thereby keeping the samples set in time.

2. The medical system of claim 1, wherein the processor is further configured to determine a heart rate based on an R-R interval of QRS complexes detected from the one or more ECG signals excluding QRS complexes that are within the blanking period.

3. The medical system of claim 1, wherein the processor is further configured to:
detect a plurality of dominant beats from the one or more ECG signals excluding the one or more ECG signals within the blanking period; and
calculate a heart rate based on an R-R interval of dominant beats.

4. The medical system of claim 1, wherein the measurement comprises a first measurement and a second measurement, and wherein the first measurement is an absolute value of an amplitude difference between consecutive samples and the second measurement is an absolute value of an amplitude difference between a first sample and a last sample in a series of samples.

5. The medical system of claim 4, wherein the noise threshold comprises a first threshold and a second threshold, wherein the first threshold is about 2 m V/sample and the second threshold is about 5 mV, and wherein the first threshold corresponds to the first measurement and the second threshold corresponds to the second measurement.

6. The medical system of claim 1, wherein the blanking period is implemented by a bi-directional filter configured to blank samples before and after the samples that exceed the noise threshold.

7. The medical system of claim 1, wherein the processor is configured to blank ECG signals having a duration of less than 30 seconds between two blanking periods.

8. A method for filtering noise from one or more ECG signals, the method comprising:
detecting, using a plurality of ECG electrodes, the one or more ECG signals;
sampling, using a noise filter module, the one or more ECG signals at a predefined frequency;
determining, using the noise filter module, if a measurement of samples exceeds a noise threshold;
providing, using the noise filter module, a blanking period if the measurement of the samples exceeds the noise threshold; and
determining a heart rate based on an R-R interval of QRS complexes detected from the one or more ECG signals excluding the QRS complexes that are within the blanking period.

9. The method of claim 8, wherein the blanking period is implemented by a bi-directional filter configured to blank samples before and after the samples that exceed the noise threshold.

10. The method of claim 8, wherein the blanking period replaces the samples in the blanking period with a not a number (NaN) value, thereby keeping the samples set in time.

11. The method of claim 8, wherein the blanking period further comprises about 250 samples to about 450 samples before and after the samples that exceed the noise threshold.

12. The method of claim 8, wherein the measurement comprises a first measurement and a second measurement, wherein the first measurement is an absolute value of an amplitude difference between consecutive samples and the second measurement is an absolute value of an amplitude difference between a first sample and a last sample in a series of samples.

13. The method of claim 12, wherein noise threshold comprises a first threshold of about 2 mV/sample and a second threshold of about 5 mV, and wherein the first threshold corresponds to the first measurement and the second threshold corresponds to the second measurement.

14. The method of claim 8, wherein determining the heart rate further comprises:
detecting a plurality of dominant beats from the one or more ECG signals excluding the one or more ECG signals within the blanking period; and
calculating the heart rate based on an R-R interval of dominant beats.

15. A non-transitory computer-readable medium, encoded with instructions for filtering noise from one or more ECG signals stored thereon that, when executed by a computing device, cause the computing device to perform operations for filtering noise from the one or more ECG signals, the operations comprising:
receiving the one or more ECG signals from a patient via a plurality of ECG electrodes;
sampling, using a noise filter module, the one or more ECG signals at a predefined frequency;
determining, using the noise filter module, if a measurement of samples exceeds a noise threshold;
providing, using the noise filter module, a blanking period if the measurement of the samples exceeds the noise threshold; and
determining a heart rate based on an R-R interval of QRS complexes detected from the one or more ECG signals excluding the QRS complexes that are within the blanking period.

16. The non-transitory computer-readable medium of claim 15, wherein the blanking period is implemented by a bi-directional filter configured to blank samples before and after the samples that exceed the noise threshold, and wherein the blanking period further comprises about 250 samples to about 450 samples before and after the samples that exceed the noise threshold.

17. The non-transitory computer-readable medium of claim 15, wherein the blanking period replaces the samples in the blanking period with a not a number (NaN) value, thereby keeping the samples set in time.

18. The non-transitory computer-readable medium of claim 15, wherein determining the heart rate further comprises:
detecting a plurality of dominant beats from the one or more ECG signals excluding the one or more ECG signals within the blanking period; and
calculating the heart rate based on an R-R interval of dominant beats.

19. The non-transitory computer-readable medium of claim 15, wherein the measurement comprises a first measurement and a second measurement, and wherein the first measurement is an absolute value of an amplitude difference between consecutive samples and the second measurement is an absolute value of an amplitude difference between a first sample and a last sample in a series of samples.

20. The non-transitory computer-readable medium of claim 19, wherein the noise threshold comprises a first threshold of about 2 m V/sample and a second threshold of about 5 mV, and wherein the first threshold corresponds to the first measurement and the second threshold corresponds to the second measurement.
